(19) United States
(12) Patent Application Publication
(10) Pub. No.: US 2025/0000932 A1
(43) Pub. Date: Jan. 2, 2025

(54) DIAGNOSIS AND TREATMENT OF VASODILATORY SHOCK

(71) Applicant: University Of Maryland, Baltimore, Baltimore, MD (US)

(72) Inventors: Michael MCCURDY, Elkridge, MD (US); Jonathan CHOW, Fox Island, WA (US); Maniraj Jeyaraju, Chapel Hill, NC (US)

(21) Appl. No.: 18/761,250

(22) Filed: Jul. 1, 2024

Related U.S. Application Data
(60) Provisional application No. 63/524,302, filed on Jun. 30, 2023.

(51) Int. Cl. A61K 38/08; A61P 9/02; C12Q 1/37; G01N 33/74

(52) U.S. Cl. CPC A61K 38/085; A61P 9/02; C12Q 1/37; G01N 33/74; G01N 2800/32

(57) ABSTRACT

Angiotensin II therapy has been found to increase mean arterial pressure in patients with catecholamine-resistant vasodilatory shock. Secondary analysis of the dataset from the Angiotensin II for the Treatment of High-Output Shock 3 (ATHOS-3) Study demonstrates that serum renin concentration is markedly elevated in some patients with catecholamine-resistant vasodilatory shock. This Invention relates to a method for identifying a subset of patients with elevated serum renin levels who gain a survival advantage when treated with angiotensin II. The invention therefore provides a method for diagnosis and treatment of catecholamine-resistant vasodilatory shock.

### CROSS-REFERENCE TO RELATED APPLICATIONS

This application is a United States utility application which claims the benefit of U.S. provisional application Ser. No. 63/524,302, filed 30 Jun. 2023. The entire contents of this provisional application is hereby incorporated by reference as if fully set forth herein.

### BACKGROUND

### 1. Field of the Invention

The invention relates to the general field of medicine and in particular to a method for diagnosis and treatment of vasodilatory shock.

### 2. Background of the Invention

Vasodilatory shock requiring vasopressor therapy is associated with poor outcomes. In particular, catecholamine-resistant vasodilatory shock (CRVS), in which hypotension persists despite the use of high-dose vasopressors, carries a 50% to 80% mortality. In such patients, first-line vasopressor therapy, which includes norepinephrine followed by epinephrine or vasopressin, may not be able to achieve target mean arterial pressure (MAP) alone. Accordingly, synthetic human angiotensin II has been approved recently for the treatment of CRVS in the United States and Europe.

Angiotensin II is an integral part of the renin-angiotensin-aldosterone system (RAAS). See FIG. 1. Angiotensin 1, the precursor of angiotensin II, is cleaved from angiotensinogen by renin, an enzyme released by the macula densa in response to sympathetic nerve activation, hypotension, or decreased sodium delivery to the distal tubule. Renin release is inhibited by high angiotensin II generation and promoted by low angiotensin II generation via a biofeedback loop involving the angiotensin type 1 receptor. As such, renin levels can be expected to be high in states of impaired angiotensin II generation. In this regard, angiotensin II is generated by angiotensin-converting enzyme (ACE), an endothelial membrane-bound enzyme that cleaves angiotensin I to angiotensin II. Thus, endothelial injury accompanying CRVS should decrease ACE function, increase angiotensin I to angiotensin II ratios. promote renin release, and potentially identify patients most likely to benefit from angiotensin II therapy. In support of this theory, high ratios of angiotensin I to angiotensin II correlate with negative outcomes in such patients. Moreover, in a study of patients with sepsis, reduced angiotensin II and ACE levels outperformed established illness-severity scores in predicting outcomes. Unfortunately, assessing ACE activity is not amenable to simple plasma measurement and measurement of the angiotensin I to angiotensin II ratio is difficult and not available in a timely manner.

### SUMMARY OF THE INVENTION

Thus, there exists a need in the art for a method of determining whether angiotensin II therapy will benefit a particular patient and treating such patients. In particular embodiments, the present invention relates to a method of diagnosing an angiotensin II-responsive patient suffering from vasodilatory shock comprising: (a) obtaining a blood, plasma, or serum sample from the patient; (b) measuring plasma renin levels in the sample to obtain a baseline renin level; (c) measuring the angiotensin I levels and the angiotensin II levels in the sample and calculating the angiotensin I/angiotension II ratio in the sample; and (d) if the baseline renin level is at least about three fold higher than a normal renin level, then diagnosing the patient with angiotensin II-responsive vasodilatory shock.

Certain additional embodiments of the invention include a method of treating vasodilatory shock comprising: (a) diagnosing the patient with angiotensin II-responsive vasodilatory shock according to the method of claim 1; and (b) treating the patient by administration of angiotensin II.

Certain additional embodiments of the invention include methods wherein if the baseline renin level is at least about three-fold higher than a normal renin level and the renin level is positively and statistically significantly correlated with angiotensin I levels, then the patient is diagnosed with angiotensin II-responsive vasodilatory shock.

Certain additional embodiments of the invention include a method of treating vasodilatory shock comprising: (a) diagnosing the patient with angiotensin II-responsive vasodilatory shock according to the method of claim 3; and (b) treating the patient by administration of angiotensin II.

In other embodiments, the vasodilatory shock is catecholamine-resistant vasodilatory shock.

In other embodiments, the administration of angiotensin II is intravenous.

### BRIEF SUMMARY OF THE DRAWINGS

Certain embodiments are illustrated by way of example, and not by way of limitation, in the figures of the accompanying drawings.

FIG. 1 is a schematic showing the renin-angiotensin-aldosterone system disturbance hypothesis. ACE refers to angiotensin-converting enzyme.

FIG. 2A, FIG. 2B, and FIG. 2C are graphs showing renin, angiotensin I, and angiotensin II correlations, including the relationship between baseline serum renin and angiotensin I/II ratio (FIG. 2A), the relationship between serum renin and angiotensin I (FIG. 2B), and the relationship between serum renin and angiotensin I at hour 3 (FIG. 2C).

FIG. 3A and FIG. 3B show serum angiotensin I and renin levels, including serum antgiotensin I levels (FIG. 3A) and serum renin levels (FIG. 3B). All values are median (interquartile range).

FIG. 4A and FIG. 4B are Kaplan-Meier survival plot according to renin levels and treatment with angiotensin II or placebo. FIG. 4A shows the day 28 survival: renin level below population median. FIG. 4B shows day 28 survival: renin level above population median. CI indicates confidence intervals; HR indicates hazard ratio; RR indicates relative risk.

FIG. 5 presents serum renin levels in patients studied in the ATHOS-3 trial compared to normal values or conditions of increased renin levels. Conversion of plasma renin activity to direct renin adopted from Hartman et al. SBP indicates spontaneous bacterial peritonitis; uRAS indicates unilateral renal artery stenosis.

FIG. 6A (514 nm) and FIG. 6B (632 nm), show the influence of excitation wavelength on initial Raman spectra.

FIG. 6C (not photobleached) and FIG. 6D (photobleached), show photobleaching effects.

FIG. 6E (*=high renin; 2=non-high renin) and FIG. 6F, show a comparison of spectra to reference literature.

### DETAILED DESCRIPTION OF EMBODIMENTS OF THE INVENTION

### 1. Overview

Serum renin is substantially elevated in most patients with CRVS, and it positively and significantly correlates with angiotensin I/II ratios. In addition, treatment with angiotensin II rapidly decreases such elevated serum renin levels. After multivariate adjustment, however, elevated serum renin levels are associated with an increased risk of death. On the other hand, exogenous synthetic human angiotensin II appears to rapidly suppress both serum renin and angiotensin I levels, lending support to the hypothesis that the RAAS is disturbed in patients with CRVS. Finally, elevated serum renin appears to identify patients who gain a survival advantage when treated with synthetic human angiotensin II. In their aggregate, the findings presented here support a novel approach to the treatment of CRVS, based biological and clinical principles.

Angiotensin II therapy increases mean arterial pressure in patients with catecholamine-resistant vasodilatory shock. This secondary analysis of the ATHOS-3 dataset demonstrates that serum renin concentration is markedly elevated in some patients with catecholamine-resistant vasodilatory shock. It also demonstrates that elevated serum renin can identify a subset of patients who gain a survival advantage when treated with angiotensin II. This finding supports a novel approach to the treatment of catecholamine-resistant vasodilatory shock.

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising,” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

### 3. Embodiments of the Invention

### A. Introduction

Certain embodiments of the invention presented here are methods for determining and treating shock. For example, certain methods include measuring plasma renin levels in patients with shock, including vasodilatory or catecholamine-resistant vasodilatory shock (CRVS), determining an angiotensin I to angiotensin II ratio (ANG I/II ratio), and administering intravenous angiotensin II therapy. Thus, measuring high levels of renin are able to help to identify such high-risk patients with a degree of ACE dysfunction and high angiotensin I to angiotensin II ratios. Laboratory assays that measure renin levels are inexpensive, have demonstrated established performance, and can be easily set up by most laboratories; importantly, measurement of renin levels outperforms that of maximum serum lactate levels as a predictor of mortality in the intensive care unit (ICU).

For example, in one embodiment, measuring plasma renin levels can include using a Raman spectroscopy device. However, in other embodiments, measuring plasma renin levels can include using a mass spectroscopy device, an ELISA system, or the like. Furthermore, determining the ANG I/II ratio can include measuring an endogenous and exogenous angiotensin II level, as further described below, and an angiotensin I level. In another embodiment, the need for treatment can be determined using the ANG 1111 ratio by comparing the measured ANG I/II ratio to a value or range of values (such as in a table or chart, described further below). Still another embodiment includes administering treatment, such as intravenous angiotensin II therapy to a patient. In still another embodiment, the method includes causing administration of a treatment to a patient.

In some embodiments, the method includes determining at least one of endogenous and exogenous angiotensin II levels. In further embodiments, the method increases determining arterial pressure changes or effects based on at least one of angiotensin I/II ratio or angiotensin II levels. For example, one effect can be an increasing mean arterial pressure in patients with CRVS. Thus, according to many embodiments described herein, determining renin levels can identify patients most likely to benefit from therapy and further providing such therapy.

### B. Key Findings

In patients enrolled in the ATHOS-3 study, the median baseline renin level was three-fold higher than normal. Patients with baseline renin levels above the study population median had significantly higher median angiotensin 1111 ratios, and their renin levels positively and significantly correlated with such ratios and with angiotensin I levels. Moreover, patients treated with exogenous angiotensin II experienced a significantly greater percentage reduction in both serum renin levels and angiotensin I levels after 3 hours of treatment than patients in the placebo group. In addition, after adjusting for APACHE II score, MAP, and norepinephrine-equivalent dose, elevated serum renin levels were associated with an increased risk of death. Finally, in patients with a baseline serum renin concentration above the population median, treatment with angiotensin II decreased mortality when compared to placebo and was independently associated with survival after multivariable adjustment.

### C. Implications of Study Findings

This study has implications for the direct management of patients with CRVS. In this setting, the measurement of serum renin levels helps to identify patients who would be most likely to benefit greatly from angiotensin II administration. The observed response to the angiotensin II administration also can help guide the monitoring of such treatment, and offer an approach tailored to the patient's response, which can translate into greater clinical benefits overall. From a practical point of view, as renin assays are widely available and inexpensive, utilization of this biomarker for vasopressor-targeted therapy is logistically feasible. In this regard, our findings imply that lowering of renin levels together with achievement of target MAP may offer a combined biological and clinical target during angiotensin II administration. Finally, in patients with CRVS receiving advanced cardiovascular supportive care, the findings that both renin and angiotensin I are suppressible by exogenous angiotensin II provides evidence that endogenous angiotensin II insufficiency may be an important contributor to increased serum renin and angiotensin I concentrations.

### D. Additional Comments

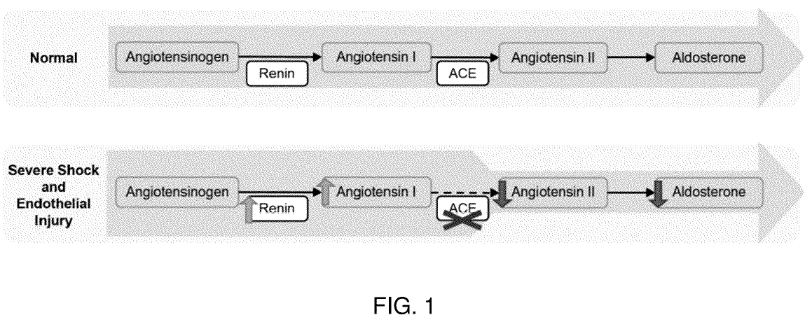

Decreased ACE activity was theorized to be a key mechanism in CRVS that is sensitive to angiotensin IL. The ATHOS-3 trial, and its analysis showed that this theory is mechanistically linked to such a RAAS-disturbance hypothesis. See FIG. 1. In addition, the findings presented here are consistent with the physiology of the RAAS and the effects of ACE inhibition. The clear reduction in both renin and angiotensin I with exogenous angiotensin II administration, compared with minimal changes in the placebo arm of the study, gives credence to the concept of decreased activity of this pathway and relative preservation of a biofeedback response. ATHOS-3 was an international, prospective, randomized, placebo-controlled trial in which adequate resuscitation per international consensus guidelines was a prerequisite for trial entry, thus increasing the generalizability of our findings.

### 4. Examples

This invention is not limited to the particular processes, compositions, or methodologies described, as these may vary. The terminology used in the description is for the purpose of describing the particular versions or embodiments only and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety; nothing herein is to be construed as an admission that the invention is not entitled to antedate such disclosure by virtue of prior invention.

### Example 1: General Methods

### A. Study Design and Patients.

The use of Angiotensin II for the Treatment of High-Output Shock 3 (ATHOS-3) study has been described by Khanna et al. and Chawla et al. In brief, adults with vasodilatory shock despite volume resuscitation with 2:25 mL/kg and high-dose vasopressors were randomly assigned 1:1 to receive synthetic human angiotensin II (La Jolla Pharmaceutical Company™) or saline placebo plus standard vasopressors. Randomization was stratified according to MAP at screening and Acute Physiology and Chronic Health Evaluation II (APACHE II) score.

The study drug infusion started at 20 ng/kg/min and was adjusted during the first 3 hours to increase the MAP to 2:75 mm Hg while keeping other vasopressors constant. Thereafter, the study drug and other vasopressors were adjusted at the discretion of the treating ICU team to maintain a MAP between 65 and 75 mm Hg. At 48 hours, the infusion was discontinued according to a protocol-specified tapering process. However, continuation was allowed for up to 7 days at the discretion of the ICU team.

### B. Ethics Approval and Consent to Participate.

The study, inclusive of the sample collection, was conducted in accordance with Good Clinical Practice guidelines, applicable local regulations, and the ethical principles described in the Declaration of Helsinki. The protocol, informed consent form, and all other documents were reviewed and approved by the respective independent institutional review boards before study initiation.

### C. Serum Renin, Angiotensin 1, and Angiotensin II Measurement.

Serum concentrations of renin, angiotensin I, and angiotensin II were measured after randomization but prior to administration of the study drug and at 3 hours after initiation of study drug.

### D. Statistical Analysis.

Descriptive statistics with 95% confidence intervals (Cls) were use to summarize data according to treatment group. Differences between treatment groups were analyzed using the Wilcoxon rank-sum test or analysis of variance for continuous or ordinal variables and the chi-square or Fisher's exact test for discrete variables. Time-to-event data, including survival, was summarized using Kaplan-Meier estimates and comparing them by log-rank test. Hazard ratios (HRs) were estimated from a proportional hazards model. A two-sided alpha level of 0.05 was used to test for differences in treatment outcomes without adjustments for multiplicity.

Correlation coefficients were calculated for renin versus angiotensin I and the angiotensin I/II ratio. Correlation coefficients were also calculated for renin versus angiotensin I for hour 0 (baseline) and hour 3. For multivariate analyses, covariates that were utilized for study stratification (Model A), covariates that were different between groups defined by P<0.10 (Model B), and statistically significant covariates from Models A and B were included in a multivariate logistic regression (Model C). The dichotomized baseline covariates that were used for study stratification were MAP<65 mm Hg and APACHE II score>30.

Safety was evaluated by assessment of treatment-emergent adverse events, serious adverse events, and adverse event-related drug discontinuations. These data were analyzed with the use of SAS software, version 9.4 (SAS Institute™, Cary, NC).

### Example 2: Study Results

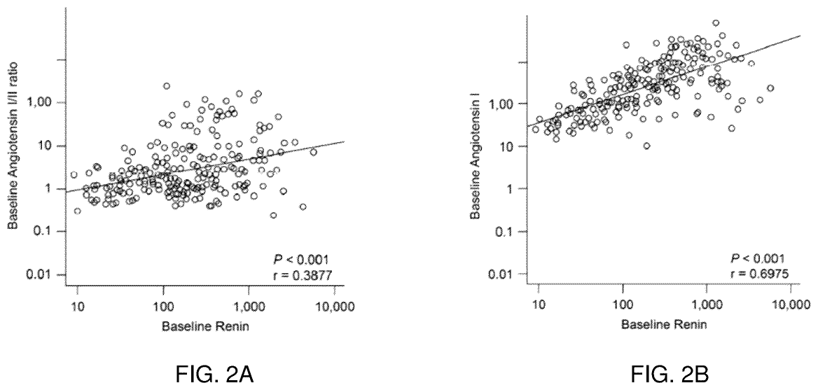

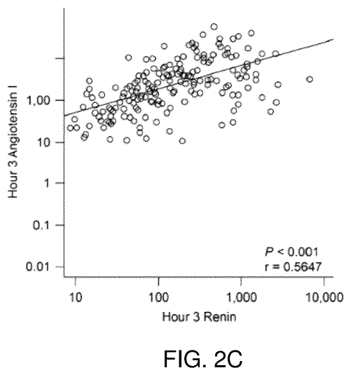

Of the 321 patients with CRVS studied in ATHOS-3, 255 (79.4%) had suitable serum samples available for analysis of renin concentrations at baseline (hour 0). In these patients, baseline serum renin concentrations were comparable between the angiotensin II and placebo arms and were elevated to levels above the upper limit of normal in 197 (77%) patients. The median serum renin concentration of the entire population was 172.7 pg/mL (interquartile range [IQR]: 60.7-440.6 pg/mL); this was approximately 3 times the upper limit of normal and exceeded renin levels seen in syndromes associated with particularly high levels. See FIG. El-5. As expected, baseline serum renin levels were positively correlated with the baseline angiotensin 1/angiotensin II ratio (P<0.001) and angiotensin I (P<0.001). In addition, renin and angiotensin I were also positively correlated at 3 hours of treatment (P<0.001). See FIG. 2.

Tables 1 and 2, below, show patient demographic and clinical data dichotomized by median serum renin levels. At baseline, patients with serum renin levels above the population median were similar to those with levels below the population median, except for baseline levels of angiotensin 1, angiotensin II, angiotensin I/II ratio, and norepinephrine-equivalent dose.

TABLE 1
Demographics of Study Patients.

| | Renin Below | | Renin Above | | |
|---|---|---|---|---|---|
| | Population Median | | Population Median | | |
| | N = 127 | | N = 128 | | P Value |
| Age, years, median (IQR) | 63 | (51-74) | 62 | (51-74) | 0.5192* |
| Gender | 48 | (37.8%) | 52 | (40.6%) | 0.7008** |
| Female | 79 | (62.2%) | | | |
| Male | | | 76 | (59.4%) | |
| Race | 105 | (82.7%) | 100 | (78.1%) | 0.4308** |
| White | | | | | |
| Nonwhite | 22 | (17.3%) | 28 | (21.9%) | |
| Baseline albumin (g/dL) | 2.2 | (1.8-2.8) | 2.3 | (1.9-2.7) | 0.6115* |
| Median (IQR) | | | | | |
| Baseline MELD | 21 | (14-25) | 23 | (18-27) | 0.0681* |
| Median (IQR) | | | | | |
| Baseline MAP (mm Hg) | 66.3 | (63.7-68.3) | 66.3 | (62.9-68.7) | 0.6779* |
| Median (IQR) | | | | | |
| Baseline APACHE II score | 27 | (21-33) | 29 | (24-35) | 0.0691* |
| Median (IQR) | | | | | |
| Medical history, ARDS | 107 | (84.3%) | 101 | (78.9%) | 0.3328** |
| No | 20 | (15.7%) | 27 | (21.1%) | |
| Yes | | | | | |

APACHE = Acute Physiology and Chronic Health Evaluation ; ARDS = acute respiratory distress syndrome; IQR, interquartile range; MAP = mean arterial pressure; MELD = Model for End-stage Liver Disease ; NED = norepinephrine equivalent dose. ACE inhibitor exposure was determined by the presence of an ACE inhibitor in the medical chart within 7 days prior to study enrollment.
*Wilcoxon rank-sum test.
**Fisher 's exact test.

TABLE 2
Clinical Characteristics of Study Patients.

| | Renin Below | | Renin Above | | |
|---|---|---|---|---|---|
| | Population Median | | Population Median | | |
| | N = 127 | | N = 128 | | P Value |
| Chest X-ray, ARDS | 92 | (73.0%) | 87 | (68.0%) | 0.4108** |
| No | | | | | |
| Yes | 34 | (27.0%) | 41 | (32.0%) | |
| Medical history, sepsis | | | | | 0.2697** |
| No | 21 | (16.5%) | 29 | (22.7%) | |
| Yes | 106 | (83.5%) | 99 | (77.3%) | |
| Baseline NED (ug/kg/min) Median (IQR) | 0.30 | (0.22-0.50) | 0.38 | (0.25-0.60) | 0.0132* |
| Angiotensin I (pg/ml) | 95.5 | (39.2-275) | 631 | (267-1310) | <0.0001* |
| Angiotensin II (pg/ml) | 64.6 | (24.5-160) | 139.5 | (28.2-513.5) | 0.0008 |
| Baseline angiotensin I/II ratio Median | 1.29 | (0.83-2 .59) | 3.10 | (1.17-11.90) | <0.0001* |
| (IQR) | | | | | |

APACHE = Acute Physiology and Chronic Health Evaluation; ARDS = acute respiratory distress syndrome; IQR, interquartile range; MAP = mean arterial pressure; MELD = Model for End-stage Liver Disease; NED = norepinephrine equivalent dose. ACE inhibitor exposure was determined by the presence of an ACE inhibitor in the medical chart within 7 days prior to study enrollment.
*Wilcoxon rank-sum test.
**Fisher 's exact test.

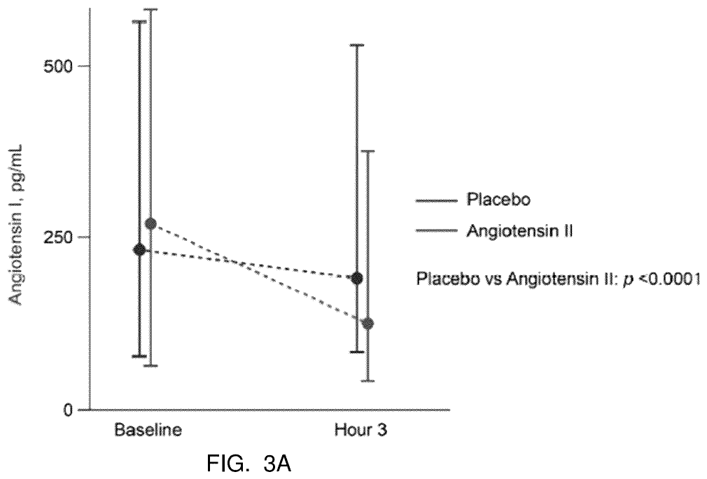

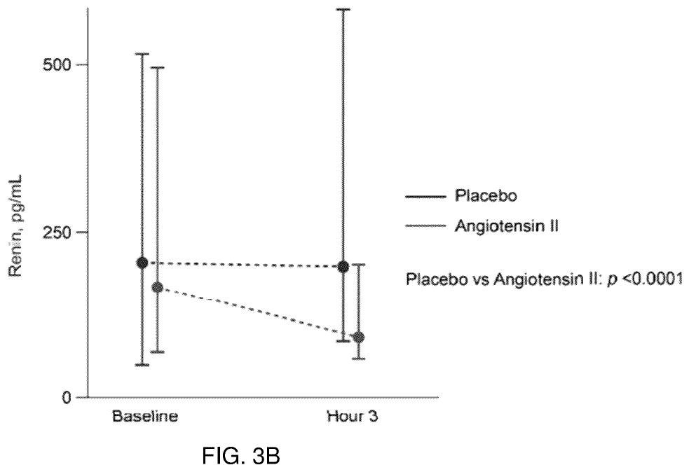

FIG. 3 shows angiotensin I and serum renin levels in patients who had samples available at baseline and at 3 hours. Patients treated with synthetic angiotensin II experienced a median reduction of 54.3% (IQR: 37.9%-66.5% reduction) in renin concentration compared with a median reduction of 14.1%0 (IQR: 37.6% n reduction to 5.1% increase) with placebo (P<0.0001). In addition. patients treated with synthetic angiotensin II experienced a 39.7% median reduction (IQR: reduction of 11.8% to reduction of 56.9%) in angiotensin I levels compared with a median reduction of 7.0% G reduction (IQR: reduction 26.7% to 20% increase) with placebo (P<0.0001). See Table 3 and Table 4, below for additional data.

TABLE 3
Numerical Data, FIG. 3A.

| | | | |
|---|---|---|---|
| Angiotensin I | Placebo | Angiotensin II | P |

| (pg/mL) | | (pg/mL) | | (pg/mL) | Value |
|---|---|---|---|---|---|
| Hour 0 | 238 | (75-653) | 260 | (72-579) | 0.66 |
| Hour 3 | 218 | (76-553) | 133 | (14-383) | 0.04 |
| Difference | −10.0 | (−78.5 to 42.1) | −76.7 | (−264.5 to 12.6) | <0.0001 |
| Difference (%) | −7.0 | (−26.7 to 20.0) | −39.7 | (−56.9 to −11.8) | <0.0001 |

TABLE 4
Numerical Data, FIG. 3B.

| | | | |
|---|---|---|---|
| | | | P |
| Renin (pg/mL) | Placebo | Angiotensin II | Value |

| | | | | | |
|---|---|---|---|---|---|
| Hour 0 | 193.7 | (58.1 to 489.8) | 146.1 | (62.4 to 412.2) | 0.42 |
| Hour 3 | 187.9 | (62.3 to 562.2) | 85.0 | (52.3 to 189.1) | 0.002 |
| Difference | −15.9 | (−72 to 13.7) | −98.9 | (−278.9 to −27.4) | <0.0001 |
| Difference % | −14.1 | (−37.6 to 5.1) | −54.3 | (−66.5 to −37.9) | <0.0001 |

### Example 3: Survival by Baseline Serum Renin Levels

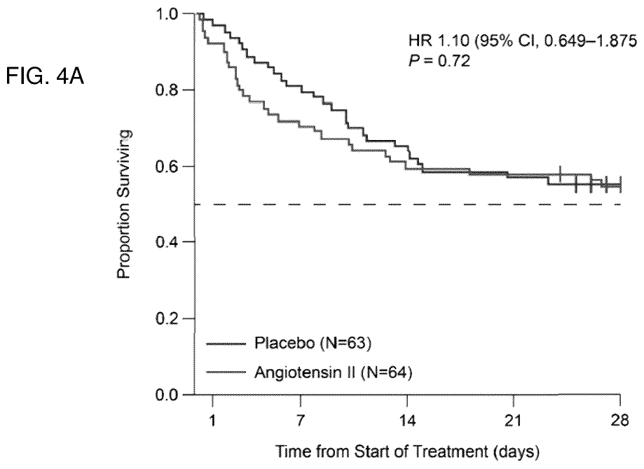

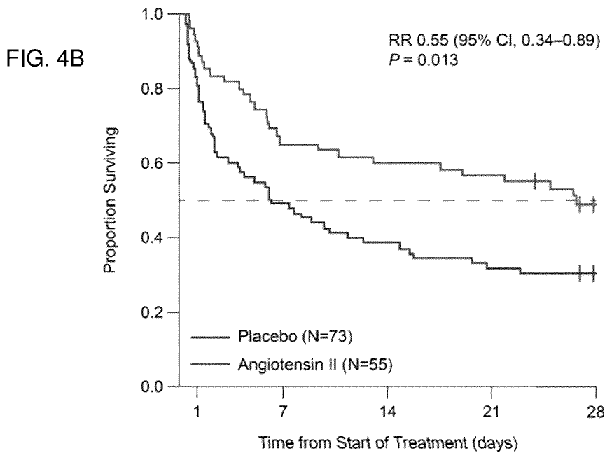

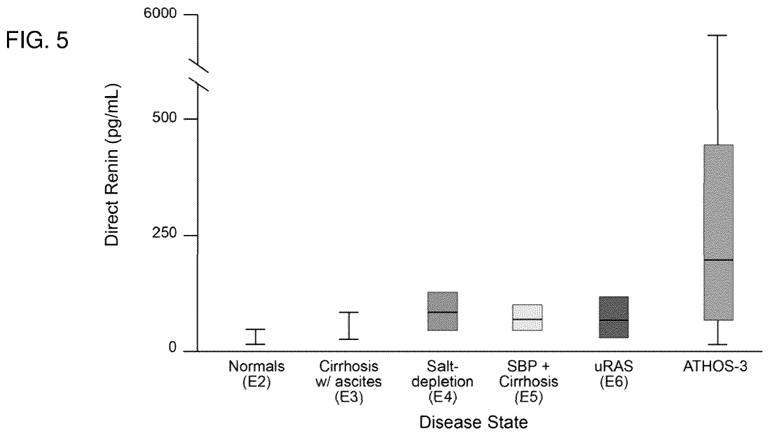

For patients with renin levels above the population median, baseline characteristics were well balanced between the placebo and angiotensin IT treatment arms. See Table 3, below. However, those treated with placebo had a 28-day mortality rate of 69.9% compared with 50.9% in those treated with angiotensin II (unadjusted HR. 0.55; 95% CI, 0.34-0.89: P=0.013). See FIG. 4. In contrast, there was no statistically significant difference in 28-day mortality for those with serum renin levels below the population median (FIG. 4).

Multivariate logistic regression within the placebo treatment arm alone showed that, after adjusting for age, sex, APACHE II score, MAP, and norepinephrine-equivalent dose, elevated serum renin levels were independently associated with an increased risk of death (HR. 2 0.11; 95% CI, 1.33-3.36; P=0.0016 (Table 3). Moreover, in patients with a renin level above the population median, multivariable analysis identified treatment with angiotensin UI as associated with decreased risk of mortality (HR, 0.62; 95% CI, 0.39-0.98; P=0.0423). See Table 5 and Table 6, below.

TABLE 5
Demographics of Patients with Serum Renin Concentrations Above
the Population Median According to Treatment Allocation.

| | Treatment with | | Treatment with | | |
|---|---|---|---|---|---|
| | Placebo | | Angiotensin II | | |
| | N = 73 | | N = 55 | | PValue |
| Age, years, median (IQR) | 63 | (51-75) | 62 | (50-72) | 0.6630* |
| Gender | | | | | |
| Female | 26 | (35.6%) | 26 | (47.3%) | |
| Male | 47 | (64.4%) | 29 | (52.7%) | 0.2064** |
| Race | | | | | |
| White | 54 | (74.0%) | 46 | (83.6%) | |
| Nonwhite | 19 | (26.0%) | 9 | (16.4%) | 0.2043** |
| Baseline | 92.8 | (75.3-110.2) | 80.4 | (70.0-106.9) | 0.1223* |
| weight | | | | | |
| Baseline | 171 | (165-179.5) | 170 | (160-180) | 0.3155* |
| height | | | | | |
| Baseline BMI | 31.1 | (25.4-39.7) | 29.2 | (24.2-37.2) | 0.2833* |
| (kg/m2) | | | | | |
| Baseline albumin | 2.3 | (1.9-2.7) | 2.3 | (1.9-2.7) | 0.7457* |
| (g/dL) ian | | | | | |

APACHE = Acute Physiology and Chronic Health Evaluation; ARDS = acute respiratory distress syndrome; BMI = body mass index; IQR = interquartile range.

TABLE 6
Clinical Characteristics of Patients with Serum Renin Concentrations
Above the Population Median According to Treatment Allocation.

| | Treatment with | | Treatment with | | |
|---|---|---|---|---|---|
| | Placebo | | Angiotensin II | | |
| | N = 73 | | N = 55 | | PValue |
| Baseline | 23 | (20-28) | 22 | (18-26) | 0.0914* |
| MELD | | | | | |
| Baseline MAP | 66.3 | (62.3-68.0) | 66.7 | (63.3-69.7) | 0.2829* |
| (mm Hg) | | | | | |
| Baseline APACHE II | 31 | (25-36) | 28 | (22-34) | 0.1437* |
| score Median | | | | | |
| Medical history, ARDS | | | | | |
| No | 55 | (75.3%) | 46 | (83.6%) | 0.2819** |
| Yes | 18 | (24.7%) | 9 | (16.4%) | |
| Medical history, sepsis | | | | | |
| No | 16 | (21.9%) | 13 | (23.6%) | |
| Yes | 57 | (78.1%) | 42 | (76.4%) | 0.8341** |
| Baseline NED | 0.40 | (0.29-0.69) | 0.36 | (0.23-0.50) | 0.0595* |
| (ug/kg/min) | | | | | |
| Angiotensin I (pg/ml) | 602 | (238-1110) | 655 | (304.5-1375) | 0.4548* |
| Angiotensin II (pg/ml) | 108 | (16.7-523) | 151 | (41.4-439) | 0.4557* |
| Baseline angiotensin I/II | | | | | 0.9573* |
| ratio Median (IQR) | 3.41 | (1.17-10.39) | 3.01 | (1.17-12.43) | |

APACHE = Acute Physiology and Chronic Health Evaluation; ARDS = acute respiratory distress syndrome; BMI = body mass index; IQR = interquartile range; MAP = mean arterial pressure; MELD = Model for End-Stage Liver Disease; NED = norepinephrine equivalent dose.
*Wilcoxon rank-sum test.
**Fisher 's exact test.

In patients with elevated renin concentrations at baseline, there was no significant difference in adverse events or serious adverse events between the angiotensin II and placebo groups (see Table 5, below).

### Example 4: Data Comparisons

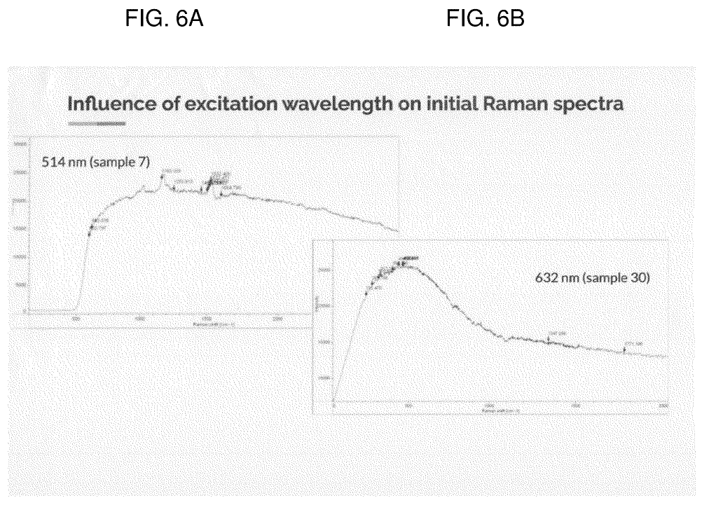

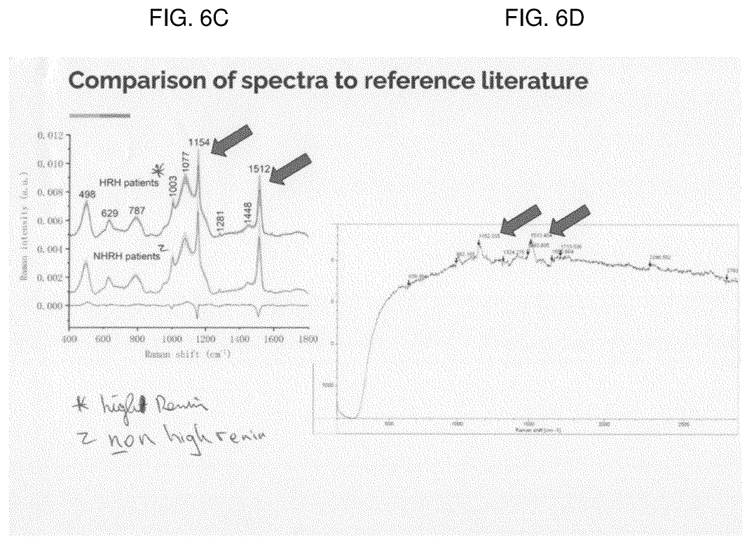

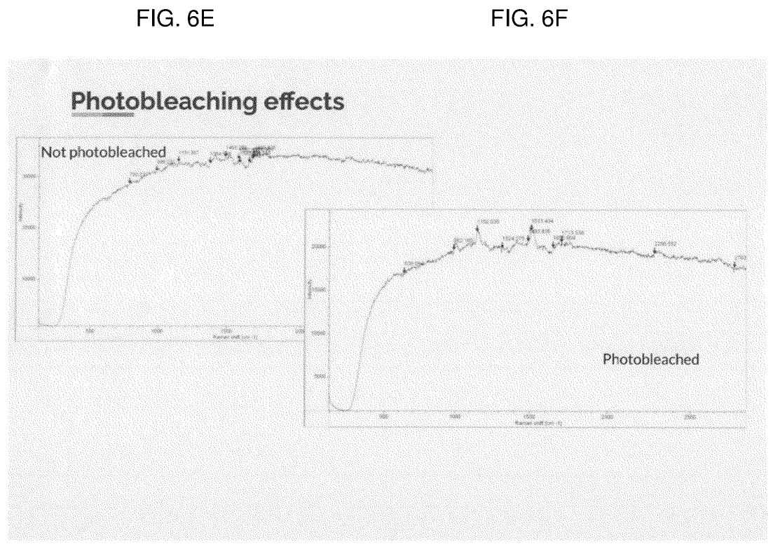

See FIG. 6A and FIG. 6B, which show the influence of excitation wavelength on initial Raman spectra.

See also FIG. 6C and FIG. 6D, which show photobleaching effects.

See also FIG. 6E and FIG. 6F, which show a comparison of spectra to reference literature.

### REFERENCES

All references listed below and throughout the specification are hereby incorporated by reference in their entirety.
1. Lambden S, Creagh-Brown B C, Hunt J, Summers C, Forni L G. Definitions and pathophysiology of vasoplegic shock. Crit Care 2018 22:174.
2. Sterling S A, Puskarich M A. Shapiro N I, Trzeciak S, Kline J A, Summers R L, Jones A E, Emergency Medicine Shock Research Network (EMSHOCKNET). Characteristics and outcomes of patients with vasoplegic versus tissue dysoxic septic shock. Shock 2013; 40:11-14.
3. Brown S M, Lanspa M J, Jones J P, Kuttler K G, Li Y, Carlson R, Miller R R 3rd, Hirshberg E L, Grissom C K, Morris A H. Survival after shock requiring high-dose vasopressor therapy. Chest 2013; 143:664-671.
4. Rhodes A, Evans L E, Alhazzani W, Levy M M, Antonelli M, Ferrer R, Kumar A, Sevransky J E, Sprung C L, Nunnally M E, Rochwerg B, Rubenfeld G D, Angus D C, Annane D, Beale R J, Bellinghan G J, Bernard G R, Chiche J D, Coopersmith C, De Backer D P, French C J, Fujishima S, Gerlach H, Hidalgo J L, Hollenberg S M, Jones A E, Karnad D R, Kleinpell R M, Koh Y, Lisboa T C, Machado F R, Marini J J, Marshall J C, Mazuski J E, Mcintyre L A, McLean A S, Mehta S. Moreno R P, Myburgh J. Navalesi P, Nishida O, Osborn T M, Perner A. Plunkett C M, Ranieri M, Schorr C A, Seckel M A. Seymour C W, Shieh L, Shukri K A, Simpson S Q, Singer M, Thompson B T, Townsend S R, Van der Poll T, Vincent J L, Wiersinga W J I Zimmerman J L, Dellinger R P. Surviving Sepsis Campaign: International Guidelines for Management of Sepsis and Septic Shock: 2016. Crit Care Med 2017; 45:486-552.
5. Khanna A, English S W, Wang X S, Ham K, Tumlin J, Szerlip H, Busse L W, Altaweel L, Albertson T E, Mackey C, McCurdy M T, Boldt D W, Chock S, Young P J, Krell K, Wunderink R G, Ostermann M, Murugan R, Gong M N, Panwar R, Hastbacka J, Favory R, Venkatesh B, Thompson B T, Bellomo R, Jensen J, Kroll S, Chawla L S, Tidmarsh G F, Deane A M; ATHOS-3 Investigators. Angiotensin 11 for the treatment of vasodilatory shock. N Engl J Med 2017; 377:419-430.
6. Chawla L S, Busse L, Brasha-Mitchell E, Davison D, Honiq J, Alotaibi Z, Seneff M G. Intravenous angiotensin II for the treatment of high-output shock (ATHOS trial): a pilot study. Crit Care 2014; 18:534.
7. Chawla L S, Busse L W, Brasha-Mitchell E, Alotaibi Z. The use of angiotensin II in distributive shock. Crit Care 2016; 20:137.
8. Paul M. Poyan Mehr A, Kreutz R. Physiology oflocal renin-angiotensin systems. Physiol Rev 2006; 86:747-803.
9. Correa T D, Takala J, Jakob S M. Angiotensin II in septic shock. Crit Care 2015; 19:98.
10. Crowley S D, Coffman T M. Recent advances involving the renin-angiotensin system. Exp Cell Res 2012; 318:1049-1056.
11. Orfanos S E, Armaganidis A, Glynos C, Psevdi E, Kaltsas P, Sarafidou P, Catravas J D, Dafni U G, Langleben D, Roussos C. Pulmonary capillary endothelium-bound angiotensin-converting enzyme activity in acute lung injury. Circulation 2000; 102:2011-2018.
12. Deitz D M. Swartz K R, Wright M, Murphy E, Connell R S, Harrison M W. Effects of E. coli endotoxin on rat plasma angiotensin converting enzyme activity in vitro and in vivo. Circ Shock 1987; 21:23-29.
13. Levi M, van der Poll T. Endothelial injury in sepsis. Intensive Care Med 2013; 39:1839-1842.
14. Wunderink R G, Albertson T E, Busse L W, Deane A M, Khanna A, McCurdy M T, Ostermann M, Young P J, Chavez A N, Handisides D R, Chen S. Chawla L S, Tidmarsh G F, Bellomo R. Baseline angiotensin levels and ACE effects in patients with vasodilatory shock treated with angiotensin II. Intensive Care Med Exp 2017; 5(Suppl 2):44.
15. Zhang W, Chen X, Huang L, Lu N. Zhou L, Wu G, Chen Y. Severe sepsis: low expression of the renin-angiotensin system is associated with poor prognosis. Exp Ther Med 2014; 7: 1342-1348.
16. Gleeson P J, Crippa I A, Mongkolpun W, Cavicchi F Z, Van Meerhaeghe T, Brimioulle S, Taccone F S, Vincent J L, Creteur J. Renin as a marker of tissue-perfusion and prognosis in critically ill patients. Crit Care Med 2019; 47:152-158.
17. Khanna A K. Tissue perfusion and prognosis in the critically ill-Is renin the new lactate? Crit Care Med 2019; 47:288-290.
18. Chawla L S, Russell J A, Bagshaw S M, Shaw A D, Goldstein S L, Fink M P. Tidmarsh G F. Angiotensin II for the Treatment of High-Output Shock 3 (ATHOS-3): protocol for a phase 11, double-blind, randomised controlled trial. Crit Care Resusc 2017; 19:43-49.
19. Hannula-Jouppi K, Massinen S, Siljander T, Makela S, Kivinen K, Leinonen R, Jiao H, Aitos P, Karppelin M, Vuopio J, Syrjanen J, Kere J. Genetic susceptibility to non-necrotizing erysipelas/cellulitis. PLoS One 2013; 8:e56225.
20. Dou X M, Cheng H J, Meng L, Zhou L L. Ke Y H., Liu L P, Li Y M. Correlations between ACE single nucleotide polymorphisms and prognosis of patients with septic shock. Biosci Rep 2017; 37.
21. Davenport M W, Zipser R D. Association of hypotension with hyperreninemic hypoaldosteronisn in the critically ill patient. Arch Intern Med 1983; 143:735-737.
22. du Cheyron D, Lesage A, Daubin C, Ramakers M, Charbonneau P. Hyperreninemic hypoaldosteronism: a possible etiological factor of septic shock-induced acute renal failure. Intensive Care Med 2003; 29:1703-1709.
23. Nguyen M, Denimal D, Dargent A, Guinot P G, Duvillard L, Quenot J P, Bouhemad B. Plasma renin concentration is associated with hemodynamic deficiency and adverse renal outcome in septic shock. Shock 2019; 52:e22-e30.
24. Kono T, Ikeda F, Oseko F, Imura H, Endo J. Effects of angiotensin I-converting enzyme inhibitor, S Q 14225, in nomal men. Endocrinol Jpn 1979; 26:411-418.
25. Azizi M, Guyene T T, Chatellier G, Wargon M, Menard J. Additive effects oflosartan and enalapril on blood pressure and plasma active renin. Hypertension 1997; 29:634-640.
26. Luque M, Martin P, Martell N, Fernandez C, Brosnihan K B, Ferrario C M. Effects of captopril related to increased levels of prostacyclin and angiotensin-(1-7) in essential hypertension. J Hypertens 1996; 14:799-805.
27. Chawla L S, Ostermann M, Forni L, Tidmarsh G F. Broad spectrum vasopressors anew approach to the initial management of septic shock? Crit Care 2019; 23:124.
28. Hartman D 1, Sagnella G A, Chesters C A, Macgregor G A. Direct renin assay and plasma renin activity assay compared. Clin Chem 2004; 50:2159-2161.
29. Renin ELISA [Instructions for Use]. Germany: DRG Instruments GmbH; 2019.
30. Bosch J, Arroyo V, Betriu A, Mas A, Carrilho F, Rivera F, Navarro-Lopez F, Rodes J. Hepatic hemodynamics and the renin-angiotensin-aldosterone system in cirrhosis. Gastroenterology 1980; 78:92-99.
31. MacFadyen R J, Jones C R., Doig J K, Bimbock H. Reid J L. Responses to an orally active renin inhibitor, remikiren (Ro 42-5892), after controlled salt depletion in humans. J Cardiovasc Pharmacol 1995; 25:347-353.
32. Sort P, Navasa M, Arroyo V, Aldeguer X, Planas R, Ruiz-del-Arbol L, Castells L. Vargas V, Soriano G, Guevara M, Gines P, Rodes J. Effect of intravenous albumin on renal impairment and mortality in patients with cirrhosis and spontaneous bacterial peritonitis. N Engl J Med 1999; 341:403-409.
33. Daidoji H, Tamada Y, Suzuki S, Watanabe K, Shikama T, Kikuchi Y, Kato S, Takahashi K, Fukui A, Matsui M, Yahagi T, Goto T. Plasma renin activity predicts the improvement in resistant hypertension after percutaneous transluminal renal artery angioplasty. Intern Med 2016:55:3421-3426.

1. A method of diagnosing an angiotensin II-responsive patient suffering from vasodilatory shock comprising:
(a) obtaining a blood, plasma, or serum sample from the patient;
(b) measuring plasma renin levels in the sample to obtain a baseline renin level;
(c) measuring the angiotensin I levels and the angiotensin II levels in the sample and calculating the angiotensin I/angiotension II ratio in the sample; and
(d) if the baseline renin level is at least about three fold higher than a normal renin level, then diagnosing the patient with angiotensin II-responsive vasodilatory shock.

2. A method of treating vasodilatory shock comprising:
(a) diagnosing the patient with angiotensin II-responsive vasodilatory shock according to the method of claim 1; and
(b) treating the patient by administration of angiotensin II.

3. The method of claim 1 wherein if the baseline renin level is at least about three-fold higher than a normal renin level and the renin level is positively and statistically significantly correlated with angiotensin I levels, then diagnosing the patient with angiotensin II-responsive vasodilatory shock.

4. A method of treating vasodilatory shock comprising:
(a) diagnosing the patient with angiotensin II-responsive vasodilatory shock according to the method of claim 3; and
(b) treating the patient by administration of angiotensin II.

8. The method of claim 1, wherein the vasodilatory shock is catecholamine-resistant vasodilatory shock.

9. The method of claim 2, wherein the administration of angiotensin II is intravenous.

10. The method of claim 4, wherein the administration of angiotensin II is intravenous.
